(19) United States
(12) Patent Application Publication
(10) Pub. No.: US 2012/0038886 A1
(43) Pub. Date: Feb. 16, 2012

(54) OPHTHALMOLOGIC OBSERVATION AND PHOTOGRAPHING APPARATUS

(75) Inventors: Yasuhiro Dobashi, Matsudo-shi (JP); Toshiaki Okumura, Tokyo (JP)

(73) Assignee: CANON KABUSHIKI KAISHA, Tokyo (JP)

(21) Appl. No.: 13/265,810

(22) PCT Filed: Apr. 22, 2010
(86) PCT No.: PCT/JP2010/002903

§ 371 Date: Oct. 21, 2011

(30) Foreign Application Priority Data
Apr. 30, 2009 (JP) 2009-111214

(51) Int. Cl. A61B 3/15

(52) U.S. Cl. 351/208

(57) ABSTRACT

An ophthalmologic observation and photographing apparatus capable of photographing a color image and a fluorescence image includes an illumination unit for illuminating a fundus of a subject's eye, an observation and photographing unit for observing and photographing a specified region of the illuminated subject's eye, a barrier filter insertably mounted to the observation and photographing unit and configured to transmit a fluorescence wavelength range and block excitation light, a color photographing unit including a tri-color separation filter and mounted in the observation and photographing unit, and an index projecting unit for projecting an index light to be photographed by superimposing on an image of the specified region of the subject's eye. The index light passes through the barrier filter, includes a wavelength range different from the fluorescence wavelength range, and can pass through a filter having a wavelength range different from a filter which transmits fluorescence through the most among the tri-color separation filter.

### TECHNICAL FIELD

The present invention relates to an ophthalmologic observation and photographing apparatus which is used in ophthalmological clinics and group health examination, for example.

### BACKGROUND ART

A fundus camera is known as an ophthalmologic photographing apparatus. In photography using a general fundus camera, general color photographing and fluorescence photography using a fluorescent agent injected into a vein of an examinee are well known.

In fluorescence photography, light to illuminate an ocular fundus, only a specific wavelength is required to excite a fluorescent agent and wavelength of the light other than the specific wavelength are not necessary. Therefore, the fundus camera includes a bandpass filter arranged in an illumination system optical path to prevent unnecessary light from reaching the ocular fundus of a subject's eye. At the same time, a filter that transmits only a fluorescence wavelength is arranged on a photographing system optical path not to photograph excess light other than the fluorescence. The fluorescent agent administered to the examinee by intravenous injection initially reaches a thick blood vessel of the ocular fundus by blood circulation, and gradually diffuses into thin blood vessels as time elapses as a medium period and a latter period. To observe how the fluorescent agent circulates, a moving image recording method has begun to be used.

To perform good fundus photography by a fundus camera, aligning positions of an ocular fundus and the fundus camera is required. Alignment widely used in the fundus camera is performed by projecting an alignment index on a cornea of a subject's eye and observing a positional relation both of a reflected light and a fundus image.

Thus, when a circulation state of the fluorescent agent is recorded as a moving image, the alignment index is photographed superposed on the fundus image, and a problem that a fundus region hidden by the index cannot be checked may occur. To avoid the superposition in photography, Japanese Patent Application Laid-Open No. 2-124137 discusses a photographing method in which an alignment index is projected to a fundus only for a specific time period before a fluorescent agent reaches the fundus, and after the fluorescence is emitted, the index image is attenuated or deleted.

With wide use of digital cameras in recent years, general digital cameras are widely used in fundus cameras. Further, increase in sensitivity and speed of image sensors exemplified by charge coupled device (CCD) in addition to digital cameras enables rapid improvement of a technique of fundus camera that handles an electronic image captured by an apparatus with a built-in CCD light-sensitive element.

A great feature of handling an electronic image is ease of generation of images. Each pixel of an electronic image includes red, green and blue (R, G, B) pixels and, by operating pixel values, arbitrary images can be created from an original image. Generally, a conventional fundus camera can generate a monochromatic image from an electronic image.

In the above described technique, the projection of the alignment index is limited to before a fluorescent light is emitted, and the position adjustment after a fluorescent light is emitted is performed visually by a photographer. Therefore, there is a problem that exact alignment may not be gained due to a skill of the photographer, and an optimum fundus image cannot be obtained.

In conventional photographing of visible fluorescence moving images, if a moving image is taken without projecting an alignment index for position adjustment to a fundus, there is a problem that a fundus image of good quality cannot be obtained due to difficulty in alignment. When a moving image is taken with the alignment index projected to the fundus, the alignment index is photographed together on the moving image, so that a fundus region overlapped with the index cannot be diagnosed.

### Citation List

### Patent Literature

PTL 1: Japanese Patent Application Laid-Open No. 2-124137

### SUMMARY OF INVENTION

The present invention is directed to an ophthalmologic observation and photographing apparatus which can perform position adjustment using an alignment index in visible fluorescence photography, and remove or attenuate the index when a moving image is stored or reproduced, so that a diagnosis of an ocular fundus region can be performed without being hidden by the index.

According to an aspect of the present invention, an ophthalmologic observation and photographing apparatus capable of photographing a color image and a fluorescence image includes an illumination unit configured to illuminate an ocular fundus of a subject's eye by white light or excitation light for fluorescence photography, an observation and photographing unit configured to observe and/or photograph a specified region of the subject's eye illuminated by the illumination unit, a barrier filter which is insertably mounted to the observation and photographing unit and is configured to transmit a range of fluorescence wavelengths and block the excitation light, a color photographing unit which includes a tri-color separation filter and is mounted in the observation and photographing unit, and an index projecting unit configured to project an index light to be photographed by superimposing on an image of the specified region of the subject's eye by the color photographing unit, wherein the index light which passes through the barrier filter, and includes a wavelength range different from the fluorescence wavelength range can pass through a filter which has a wavelength range different from a filter which transmits fluorescence through the most among the tri-color separation filter.

According to an ophthalmologic observation and photographing apparatus of the present invention, an image in which an alignment index superimposed on an original image is deleted or attenuated can be obtained by generating an image from which red color pixels are removed when a moving image is recorded or reproduced, so that an ocular fundus region hidden by an alignment index can be diagnosed. Accordingly, in moving image photographing, position adjustment can be performed using an alignment index regardless of skill of a photographer.

Other features and advantages of the present invention will become apparent from the following detailed description of exemplary embodiments with reference to the attached drawings. In the attached drawings, similar structures are denoted by the same reference numerals.

### BRIEF DESCRIPTION OF DRAWINGS

The accompanying drawings, which are incorporated in and constitute a part of the specification, illustrates exemplary embodiments, features, and aspects of the invention and, together with the description, serve to explain the principles of the invention.

FIG. 1 illustrates a configuration of a fundus camera according to an exemplary embodiment of the present invention.

FIG. 2 illustrates a fundus camera and a peripheral structure thereof.

FIG. 3 illustrates an operation panel.

FIG. 4A illustrates a positional relationship between an alignment index and a fundus image.

FIG. 4B illustrates a positional relationship between an alignment index and a fundus image.

FIG. 5A illustrates an image sensor and color filters.

FIG. 5B illustrates an image sensor and color filters.

FIG. 6 illustrates spectral sensitivity characteristics of color filters.

FIG. 7 illustrates settings of wavelengths of a fluorescence filter and an alignment index light source.

FIG. 8A illustrates a composite fundus image and R, G, and B separation images.

FIG. 8B illustrates a composite fundus image and R, G, and B separation images.

FIG. 8C illustrates a composite fundus image and R, G, and B separation images.

FIG. 8D illustrates a composite fundus image and R, G, and B separation images.

FIG. 9A illustrates a fundus image during photographing and diagnosis.

FIG. 9B illustrates a fundus image during photographing and diagnosis.

FIG. 10 illustrates a configuration of a 3 charge coupled device (3CCD) type image sensor.

### DESCRIPTION OF EMBODIMENTS

Various exemplary embodiments, features, and aspects of the invention will be described in detail below with reference to the drawings.

### Example 1

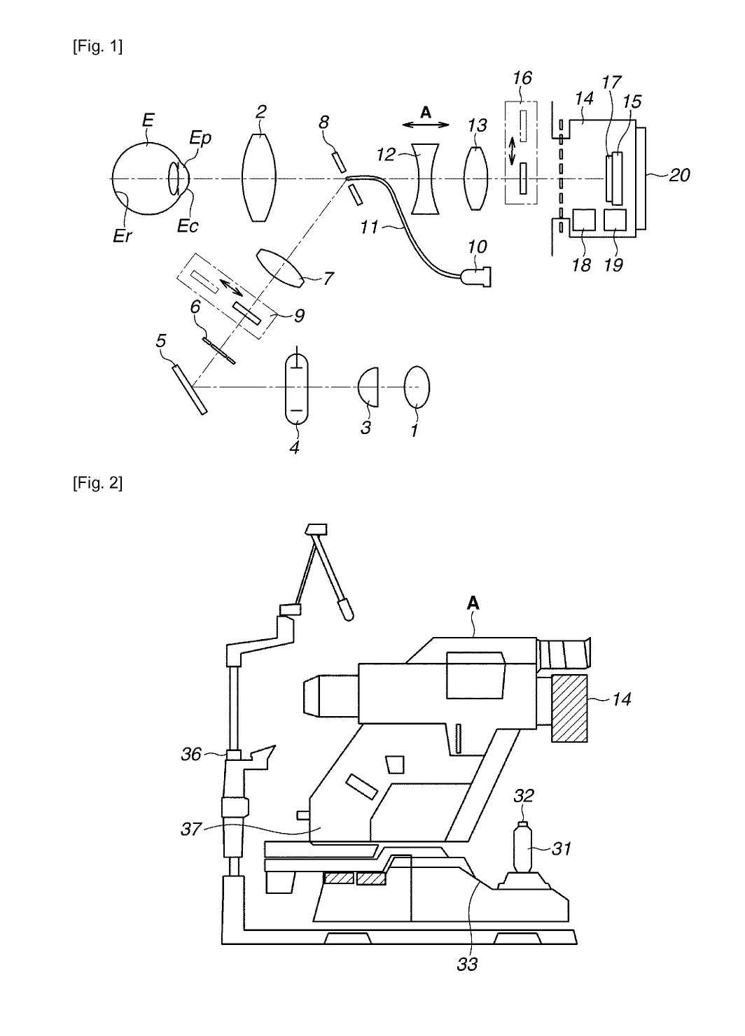

FIG. 1 illustrates a configuration of a fundus camera used in ophthalmologic observation photography according to an exemplary embodiment of the present invention. On an optical path from an observation light source 1 to an objective lens 2 placed in front of a subject's eye E, a condenser lens 3, a photographic light source 4, a mirror 5, a diaphragm 6 with a ring-shaped opening, a relay lens 7, and a perforated mirror 8 are arranged in this order. Further, an exciter filter 9 for visible fluorescence is insertably and retractably arranged between the diaphragm 6 and the relay lens 7. By those devices, a fundus illumination optical system is configured.

In a hole of the perforated mirror 8, an alignment index light source 10 for projecting an alignment index to a cornea Ep of a subject's eye is arranged via two exit ends of an optical fiber 11. On the optical path in a direction passing through the perforated mirror 8, a focusing lens 12, a photographic lens 13, and a single CCD image sensor 1 mounted in a camera body 14 are arranged. A visible fluorescence barrier filter 16 is insertably and retractably mounted between the photographic lens 13 and the camera body 14. The visible fluorescence barrier filter 16 blocks a reflected light from the ocular fundus Er and passes only a range of fluorescence wavelengths excited by the exciter filter 9. The devices described above constitute a fundus observation photographing optical system.

A tri-color separation filter 17 for color photographing is mounted in front of the image sensor 15 in the camera body 14. Moreover, an image recording unit 18 and a diagnostic image generation unit 19 are built in the camera body 14, and an image display unit 20 is mounted at the back of the camera body 14.

To the camera body 14, a plurality of types of camera, including a single-lens reflex camera can be attached, and a model with a mount of the same shape and the same focal length can be attached.

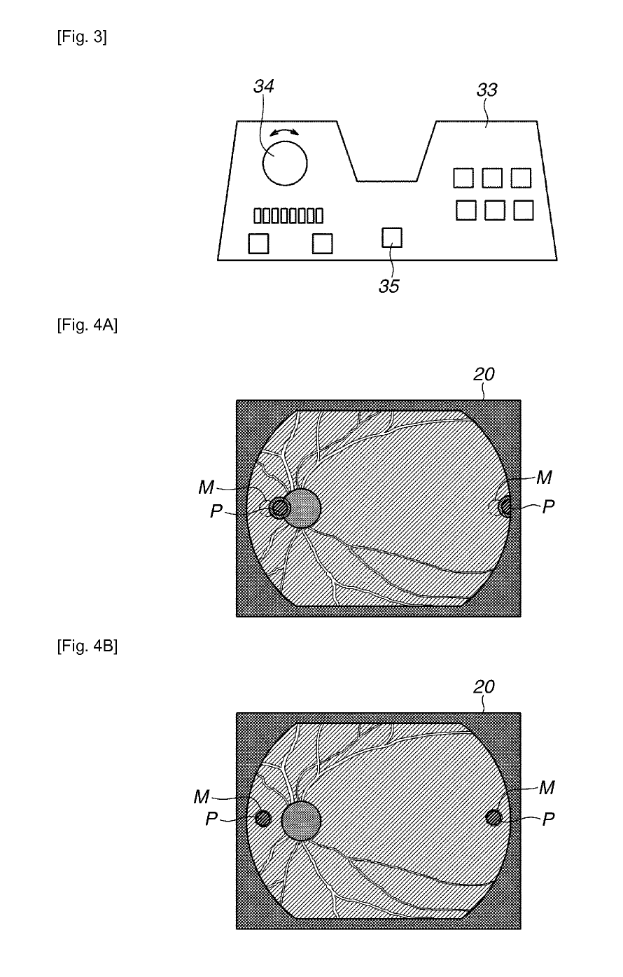

FIG. 2 illustrates a system structure. The camera body 14 can be detachably mounted on a fundus camera A, and a photographing switch 32 on a joystick 31 operated at photographing timing of the subject's eye E and an operation panel 33 of the fundus camera A are provided. As illustrated in FIG. 3, an observation light amount knob 34 for setting a photographing light amount and a timer switch 35 for starting a timer in the fundus camera A are provided on the operation panel 33.

In the fundus camera A illustrated in the exemplary embodiment, after fixing a face of the subject on a fixing base, such as a chin support 36, an examiner operates a stage unit 37 on which an optical system illustrated in FIG. 1 is mounted, projects an alignment index light emitted from the alignment index light source 10 to the ocular fundus Er, and adjust a position of the ocular fundus Er. This position adjustment is performed while observing the image display unit 20 on the camera body 14.

The alignment index light emitted from the alignment index light source 10 passes through the fiber 11 and the perforated mirror 8 and illuminates the cornea Ep. The optical system of the fundus camera A is designed so that the alignment index on an imaging plane is in focus when a distance between the fundus Er and the objective lens 2 is adequate. The exit end of the fiber 11 arranged in the hole portion of the perforated mirror 8 is split into two pieces, so that the index images can be arranged symmetric about an optical axis when a posterior pole of the fundus Er is focused on the image sensor 15.

The two alignment indexes emitted from the alignment index light source 10 are projected to the cornea Ep by an index projection unit via the objective lens 2, and a reflected light from the fundus Er passes through the objective lens 2 and forms an image on the image sensor 15 as a parallel light. A fundus image from the fundus Er illuminated by a white light emitted from the observation light source 1 similarly passes through the objective lens 2 and forms an image on the image sensor 15 as a parallel light. The examiner adjusts the position of the fundus camera A and the position of the subject's eye E by adjusting the alignment index image and the fundus image formed on the image sensor 15 to be in an adequate positional relationship with using the joystick 31 of the fundus camera A.

FIG. 4A illustrates a condition in which the alignment indexes P are out of focus and away from a proper position reference marks M in a horizontal axis Y direction and an eye axis direction Z is not in a proper position in a screen on the image display unit 20. The examiner can perform position adjustment without relying on his or her skill by operating the fundus camera A to shift a state in FIG. 4A to a state in FIG. 4B in consideration of the positional relationship between the alignment indexes P and the proper position reference marks M.

Then, the examiner, while performing the position adjustment, operates the observation light amount knob 34 on the operation panel 33, and controls the observation light source to get ready for photographing. After all preparations have been completed, a fluorescent agent is injected into a vein of the subject. At the same time, the exciter filter 9 and the barrier filter 16 are inserted into the optical path, and recording of output images from the image sensor 15 is started by the image recording unit 18. The filters 9 and 16 may be inserted by manually or automatically by interlocking with the timer switch 35 for notifying a start of photographing. Timing for a start of recording may be provided by a separately mounted recording start unit, or recording may be started in conjunction with pressing of the timer switch 35.

Moving images being recorded by the image recording unit 18 are displayed concurrently on the image display unit 20. Before the fluorescent agent flows into the fundus Er, the image display unit 20 does not display the fundus image and displays only the alignment indexes due to bandpass characteristics of the barrier filter 16. During this period, if any positional displacement occurs in vertical or horizontal directions of the subject's eye E or any change occurs in the distance between the cornea Ep and the objective lens 2, the position adjustment is performed again based on the alignment indexes P and the proper position reference marks M.

Several seconds after the fluorescent agent is intravenously administered, fluorescence begins to be observed at the fundus Er. During photographing fluorescent moving images, the examiner adjusts the observation light amount and performs position adjustment to obtain images suitable for diagnosis while watching the fundus image and the alignment index images on the image display unit 20, and continues photographing of the fluorescent moving images. The examiner performs moving image photographing for an intended time period, and completes the recording operation. An end of the recording may be provided by a separately mounted recording stop unit, as similar to the start of recording, or recording may be stopped in conjunction with pressing of the timer switch 35.

Finally, an operation for removing or attenuating only the alignment index P from the recorded moving images is performed. This operation is performed automatically using a previously set parameter in the diagnostic image generation unit 19 in the camera body 14 without drawing examiner's attention to it. Thus, the examiner can confirm the moving images from which the alignment indexes P have been removed.

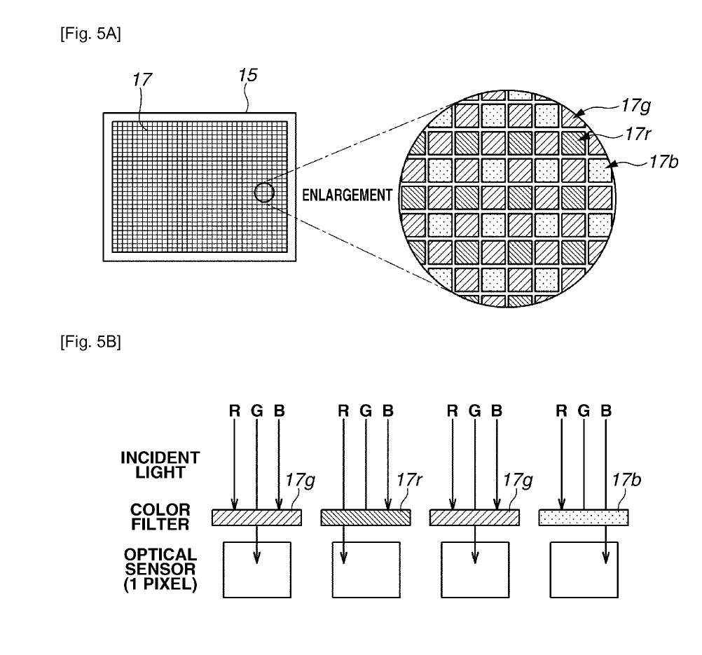

FIGS. 5A and 5B are a partially enlarged view of the image sensor 15 for color photographing and a diagram illustrating a tri-color separation filter 17 arranged on each pixel. As illustrated in FIG. 5A, a tri-color separation filter 17 which transmits only light of a specific wavelength is arranged in a mosaic form on each pixel of the image sensor 15. As illustrated in FIG. 5B, each tri-color separation filter 17 has a sensitivity characteristic to a wavelength region of light, transmits only light of a specific wavelength range, and absorbs light of other wavelengths.

The image sensor 15 is configured by a large number of optical sensor pixels, and on a light receiving surface of each pixel, a red color filter 17r, a green color filter 17g, and a blue color filter 17b of the tri-color separation filter 17 are arranged. By the filter 17r, 17g, and 17b assigning red (R), green (G), and blue (B) to each pixel, the image sensor 15 can calculate virtual pixel values for R, G, and B separation images from values of adjacent pixels, and can output a color image.

Each of the pixel values of a fundus image output from the image sensor are separated into three R, G, and B colors, and stored in the image recording unit 18 in the camera body. The diagnostic image generation unit 19 of the camera body 14 removes only an R signal or R and B signals from R, G, and B signals output from the image sensor 15, and generates an image without the R signal or the R and B signals. The image display unit 20 can display an output image from the image sensor 15 or an image generated by the diagnostic image generation unit 19 as a still image or a moving image.

In the present exemplary embodiment, visible fluorescence photography is described. However, the fundus camera can perform various types of photographing including color still image photographing by replacing the exciter filter 9 and the barrier filter 16 with filters of different characteristics, or retracting the filters from the optical path.

The most outstanding feature of the exemplary embodiment is settings of wavelength ranges of the alignment index light source 10 and the barrier filter 16 utilizing spectral sensitivity characteristics of the image sensor 15.

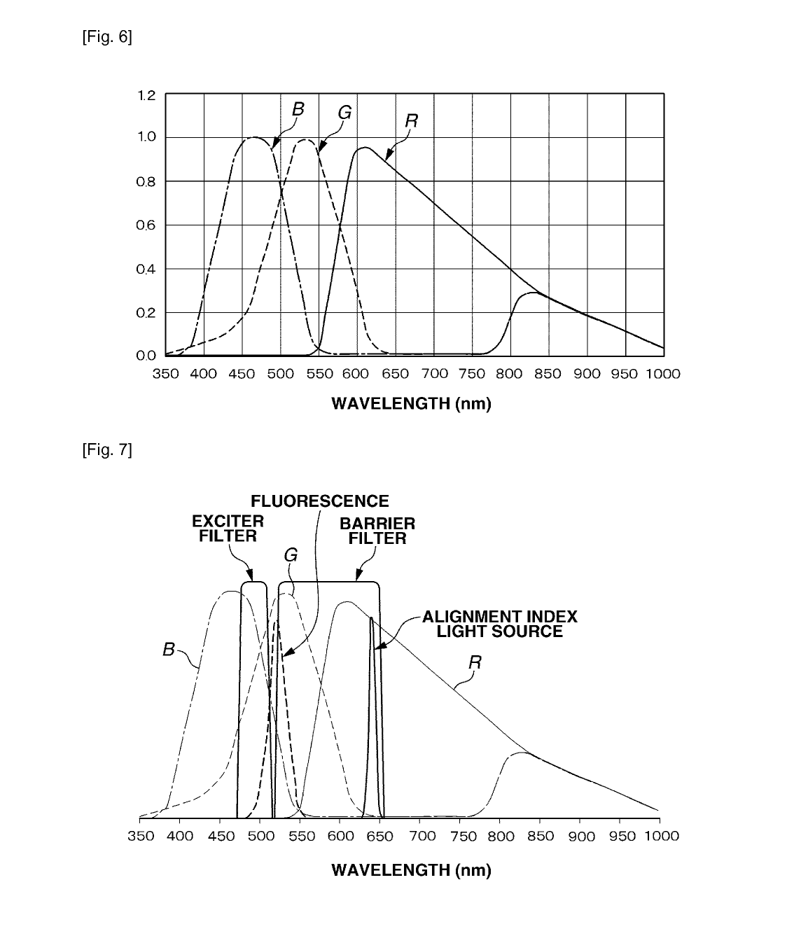

FIG. 6 illustrates a graph of spectral sensitivity characteristics when the three kinds of filters 17r, 17g, and 17b of RGB are combined with the image sensor 15. A horizontal axis denotes light wavelengths, and a vertical axis denotes sensitivity of each sensor. The graph indicates that the higher the sensitivity of a sensor, the larger the value read from the image sensor 15 becomes when the sensor receives a corresponding light wavelength. For example, from the spectral sensitivity characteristics of FIG. 6, it is understood that the characteristic G of the green filter 17g has a wavelength range of about 350 nm to 630 nm, and the sensitivity is at its maximum at a wavelength of 530 nm.

FIG. 7 illustrates characteristics of wavelength ranges and the fluorescence wavelengths of each filter and the alignment index light source 10 which are required for fluorescence photography. The thin lines indicate sensitivity characteristics of each of the filters illustrated in FIG. 6. Since the wavelength of the alignment index light source 10 necessary for the position adjustment needs to be visible, the wavelength of the alignment index light source 10 is set at a wavelength longer than that of the characteristic G of the green filter 17g and is also set to have a range of wavelengths where the green filter 17g has almost no sensitivity. In the present exemplary embodiment, the alignment index light source 10 is set at a single wavelength of 640 nm longer than the longest wavelength of 630 nm of the green filter 17g.

The exciter filter 9 for exciting a fluorescence to illuminate the fundus Er has its wavelength range set to obtain bandpass characteristics that can transmit only light of 478 nm to 515 nm wavelengths according to fluorescence characteristics of the fluorescent agent to be intravenously injected. The barrier filter 16 can transmit both the fluorescence excited at the fundus Er and the alignment light of the alignment index light source 10. The barrier filter 16 has its wavelength range set to obtain the bandpass characteristics to block light other than these two types of light.

As illustrated in FIG. 7, a fluorescence wavelength generally has wavelength characteristics with a peak at 520 nm and a short wavelength width. However, a lower limit of the bandpass characteristics of the barrier filter 16 is set at 530 nm to block an influence of an excitation light reflected from the subject's eye E. On the other hand, an upper limit is set at 650 nm to transmit the alignment light of the alignment index light source 10.

As can be seen from FIG. 7, the fluorescence wavelength emitted from fundus Er mainly passes through the green filter 17g and the blue filter 17b, and is received at the pixels corresponding to the green and blue colors on the image sensor 15. Similarly, the wavelength of the alignment index light source 10 for the alignment index passes the red filter 17r and is received at the pixels corresponding to the red color on the image sensor 15.

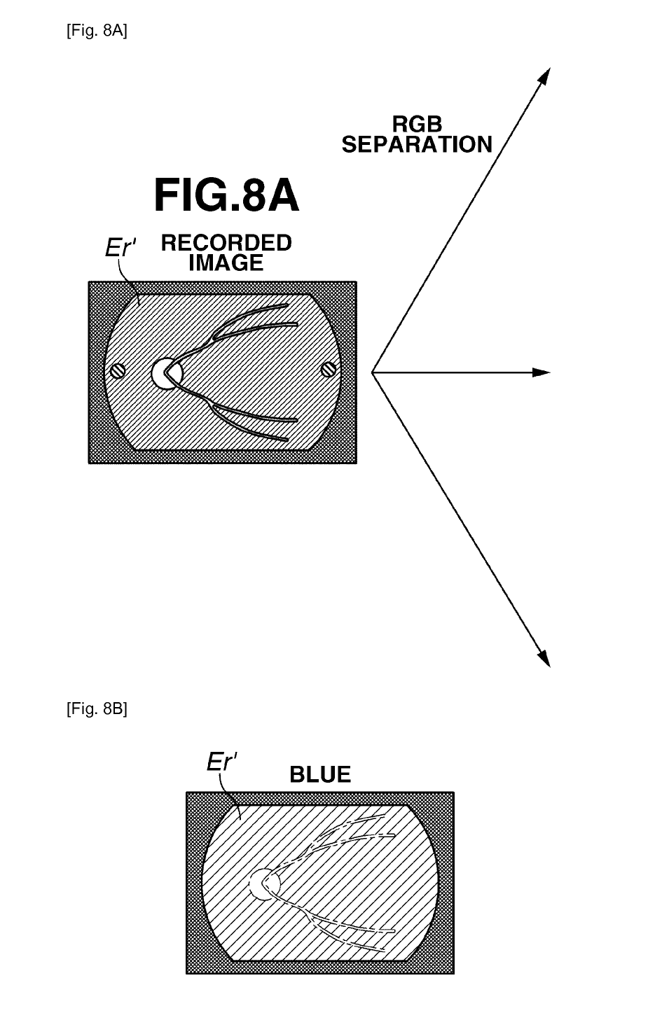

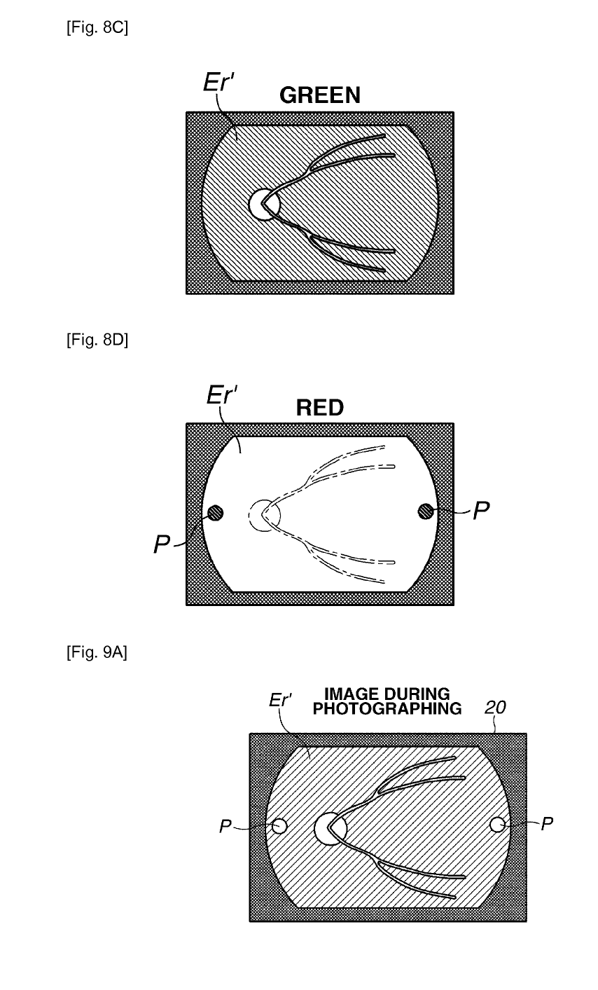

FIGS. 8A to 8D illustrate a fundus image received by the image sensor 15 and a fundus image that passes each of the filters. FIG. 8A is a composite image of the fundus images that have passed the filters. FIGS. 8B, 8C, and 8D are fundus images Er′ that pass the blue filter 17b, the green filter 17g, and the red filter 17r, respectively.

Since the wavelength of the alignment index P is set at a wavelength range longer than that of the green filter 17g, the alignment indexes P only appear on the fundus image Er′ corresponding to the red color in FIG. 8D, and are not hardly visible in the fundus images Er′ in FIGS. 8B and 8C.

Accordingly, the diagnostic image generation unit 19 removes only an image that has passed through the red filter 17r in FIG. 8D from recorded image data, and performs conversion processing to a monochromatic image to generate an image for diagnosis. More specifically, a diagnostic image is generated by performing the conversion processing to a monochromatic image using the pixels corresponding to the green color or using the pixels corresponding to the green and blue colors included in the recorded moving images. Accordingly, the alignment index P can be removed or attenuated from the generated diagnostic image, so that a specified region to be diagnosed is not hidden by the alignment index P.

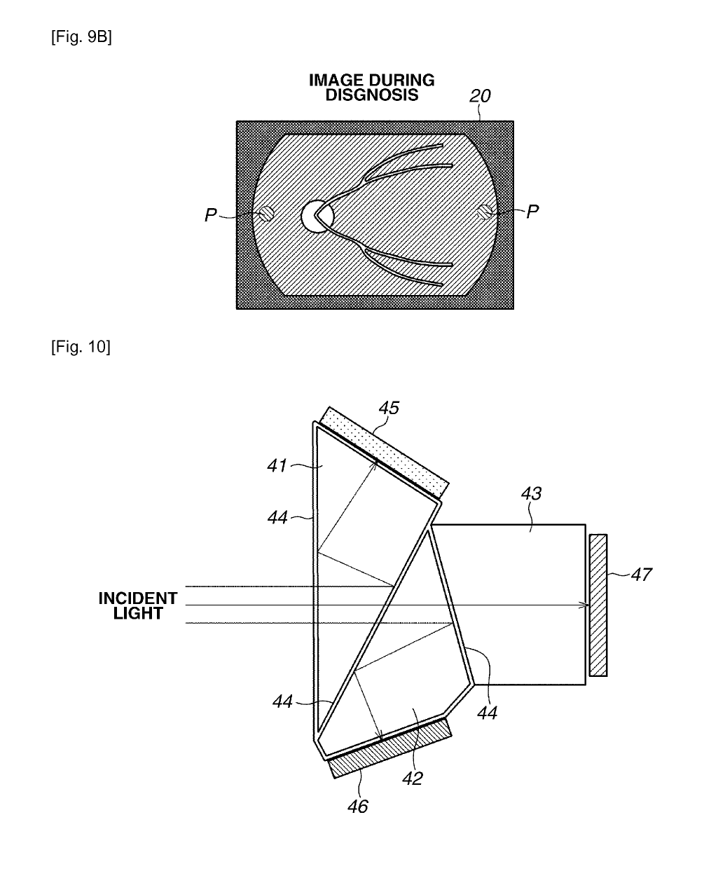

FIG. 9A illustrates a fundus image during photographing which is observed by the examiner from the image display unit 20 and allows the examiner to check the alignment indexes P and the fundus image Er′ together. FIG. 9B illustrates a fundus image during diagnosis in which the alignment indexes P are removed by the diagnostic image generation unit 19 so as not to hinder diagnosis.

Finally, the diagnostic image generated by the diagnostic image generation unit 19 may be stored again in the image recording unit 18. A focus index, a description of which is omitted, can be removed at the time of diagnosis by setting wavelengths similar to that of the alignment index.

In the exemplary embodiment, recording of moving images, image generation, and image display have been described as being performed by the image recording unit 18, the diagnostic image generation unit 19, and the image display unit 20 provided for the camera body 14. However, those operations can be performed at an information-processing equipment terminal, such as a personal computer. In such a case, output from the image sensor 15 can be transferred to an external information-processing equipment terminal by adding an image transfer unit to the camera body 14.

The positional adjustment during observation of moving images can be performed with using one or both of the image display unit 20 of the camera body 14 and a display unit of the externally connected information-processing equipment. Photographed moving images can be recorded in an image recording unit connected to the information-processing equipment. The image recording unit can be configured by a recording media, such as a hard disk, a magneto-optical (MO) disk, a Zip disk, a Jazz disk, compact disk recordable/rewritable (CD-R/RW), digital versatile disk random access memory (DVD-RAM), DVD-R/RW, and a semiconductor memory.

In the exemplary embodiment, removal of red-color images by the diagnostic image generation unit 19 is performed after moving images have been recorded. However, when images are recorded in the image recording unit 18, the moving images can be recorded in a state that the red-color images have been removed in advance by the diagnostic image generation unit 19.

Further, in the exemplary embodiment, a single CCD image sensor 15 is used. However, a 3CCD image sensor can be used. When a 3CCD image sensor is used, a dispersing prism is arranged in front of the image sensor instead of the tri-color separation filter 17.

As illustrated n FIG. 10, Surfaces of the dispersing prisms 41, 42, and 43 are provided with dichroic films, and light can be reflected and separated into three primary colors R, G, and B of light. Images sensors 45, 46, and 47 corresponding to R, G, and B are arranged on respective surfaces of the dispersing prism, and outputs form each of the image sensors 45 to 47 are introduced into the diagnostic image generation unit 19 to generate diagnostic images.

While the present invention has been described with reference to exemplary embodiments, it is to be understood that the invention is not limited to the disclosed exemplary embodiments. The scope of the following claims is to be accorded the broadest interpretation so as to encompass all modifications, equivalent structures, and functions.

This application claims priority from Japanese Patent Application No. 2009-111214 filed Apr. 30, 2009, which is hereby incorporated by reference herein in its entirety.

1. An ophthalmologic photographing apparatus according to claim 6, further comprising:
an illumination optical system configured to illuminate an ocular fundus of the subject's eye by white light or excitation light for fluorescence photography; and
an observation and photographing optical system configured to observe and/or photograph a specified region of the subject's eye illuminated by the illumination unit,
wherein the barrier filter is insertably mounted into the observation and photographing optical system and is configured to transmit a range of fluorescence wavelengths and block the excitation light,
wherein the image sensor includes a tri-color separation filter and outputs a color image,
wherein the emission projects the alignment index light to be photographed by superimposing on an image of the specified region of the subject's eye by the image sensor; and
wherein the alignment index light which passes through the barrier filter, and includes a wavelength range different from the fluorescence wavelength range can pass through a filter which has a wavelength range different from a filter which transmits fluorescence through the most among the tri-color separation filter.

2. The ophthalmologic observation and photographing apparatus according to claim 1,
wherein the generation unit generates an image by removing at least one of colors separated by the tri-color separation filter.

3. The ophthalmologic photographing apparatus according to claim 2, wherein the image sensor includes a red color filter, a green color filter, and a blue color filter.

4. The ophthalmologic photographing apparatus according to claim 3, wherein the index light has a wavelength that passes the red color filter and scarcely passes the green color filter,
wherein the image sensor outputs a fluorescence image by using all outputs of pixels of the image sensor or outputs of the pixels corresponding to the green color filter and the red color filter, and
wherein the generation unit generates a diagnostic fluorescence image by using outputs of the pixels corresponding to the green color filter or outputs of the pixels corresponding to the blue color filter and the green color filter.

5. The ophthalmologic photographing apparatus according to claim 4, wherein the image sensor includes a tri-color RGB separation filter on each pixel of a single image sensor which can output a moving image and a still image to calculate virtual pixel values of R, G, and B separation images from adjacent pixels, and generates the image data.

6. An ophthalmologic photographing apparatus capable of photographing a fluorescence image of a subject's eye, comprising:
an emission unit configured to emit alignment index light;
a barrier filter configured to pass fluorescence from the subject's eye and the alignment index light; and
an image sensor configured to separate the light passed through the barrier filter into a plurality of wavelength ranges, and to obtain electric signal for each of the separated light;
wherein a wavelength range of the barrier filter includes a plurality of the wavelength ranges which the image sensor separates the light into, and
wherein a peak wavelength of the fluorescence and a peak wavelength of the alignment index light are included in different wavelength ranges of the image sensor each other.

7. An ophthalmologic photographing apparatus according to claim 6, further comprising:
a generation unit configured to generate a image from the electric signal obtained by the image sensor by attenuating electric signal separated into the wavelength range which includes the peak wavelength of the alignment index light.

8. The ophthalmologic photographing apparatus according to claim 6, wherein
the image sensor photographs a color image while the barrier filter is retracted from an optical path of the illumination optical system, and photographs a fluorescence image while the barrier filter is inserted into the optical path.

9. The ophthalmologic photographing apparatus according to claim 6, further comprising:
a light source configured to illuminate the subjects's eye; and
an exciter filter insertably and retractably arranged on an optical path between the light source and the subject's eye,
wherein the wavelength range of the light passed through the exciter filter and the wavelength range of the fluorescence are partially overlapped, and the wavelength range of the exciter filter and the wavelength range of the barrier filter are not overlapped.

10. The ophthalmologic photographing apparatus according to claim 6, further comprising:
a switch to start photographing with the image sensor.

11. The ophthalmologic photographing apparatus according to claim 6, wherein the barrier filter is inserted into the optical path between the image sensor and the subject's eye, corresponding to the pressing of the switch.

12. The ophthalmologic photographing apparatus according to claim 6, further comprising:
a storage unit configured to start storing a moving image from the image sensor, corresponding to the pressing of the switch.

13. The ophthalmologic photographing apparatus according to claim 6, further comprising:
a display unit configured to display a moving image from the image sensor by photographing fluorescent image, and to superimpose an alignment index projected by the emission unit on the moving image while photographing fluorescent image.

14. The ophthalmologic photographing apparatus according to claim 7, comprising:
a display unit configured to display a image generated by the generation unit.
